(19) United States
(12) Patent Application Publication
(10) Pub. No.: US 2009/0082342 A1
(43) Pub. Date: Mar. 26, 2009

(54) N-SUBSTITUTED PIPERIDINE DERIVATIVES AS SEROTONIN RECEPTOR AGENTS

(75) Inventors: Henriette Kold Uldam, Vaerloese (DK); Mikkel Boas Thygesen, Fredensborg (DK)

Correspondence Address: KNOBBE MARTENS OLSON & BEAR LLP, 2040 MAIN STREET, FOURTEENTH FLOOR, IRVINE, CA, 92614, US

(73) Assignee: Acadia Pharmaceuticals, Inc., San Diego, CA (US)

(21) Appl. No.: 12/234,582

(22) Filed: Sep. 19, 2008

Related U.S. Application Data
(60) Provisional application No. 60/974,426, filed on Sep. 21, 2007.
(60) Provisional application No. 61/050,976, filed on May 6, 2008.

(51) Int. Cl. A61K 31/4468; C07D 211/58; A61K 31/5513; A61P 25/00

(52) U.S. Cl. 514/221; 546/244; 514/329

(57) ABSTRACT

Disclosed herein are isolated forms of the compounds of Formula (I), (II), (III), (IV) and (V), or a pharmaceutically acceptable salt, prodrug, hydrate, solvate, polymorph, or ester thereof. Also disclosed are methods of inhibiting an activity of a serotonin receptor, methods inhibiting an activation of a serotonin receptor, and methods of alleviating or treating various disease conditions and side effects.

### RELATED APPLICATION INFORMATION

This application claims priority to U.S. Provisional Application Ser. Nos. 60/974,426, entitled “N-SUBSTITUTED PIPERIDINE DERIVATIVES AS SEROTONIN RECEPTOR AGENTS,” filed on Sep. 21, 2007; and 61/050,976 “CO-ADMINISTRATION OF PIMAVANSERIN WITH OTHER AGENTS,” filed May 6, 2008; both of which are incorporated herein by reference in their entireties, including any drawings, for all purposes.

### BACKGROUND

1. Field

The present application relates to the fields of chemistry and medicine. More particularly, the present application relates to selective serotonin inverse agonists and/or antagonists and methods of treating diseases and/or conditions with the select selective serotonin inverse agonists and/or antagonists.

2. Description of the Related Art

Serotonin or 5-hydroxytryptamine (5-HT) plays a significant role in the functioning of the mammalian body. In the central nervous system, 5-HT is an important neurotransmitter and neuromodulator that is implicated in such diverse behaviors and responses as sleeping, eating, locomotion, perceiving pain, learning and memory, sexual behavior, controlling body temperature and blood pressure. In the spinal column, serotonin plays an important role in the control systems of the afferent peripheral nociceptors (Moulignier, Rev. Neurol. 150:3-15, (1994)). Peripheral functions in the cardiovascular, hematological and gastrointestinal systems have also been ascribed to 5-HT. 5-HT has been found to mediate a variety of contractile, secretory, and electrophysiologic effects including vascular and nonvascular smooth muscle contraction, and platelet aggregation. (Fuller, Biology of Serotonergic Transmission, 1982; Boullin, Serotonin In Mental Abnormalities 1:316 (1978); Barchas, et al., Serotonin and Behavior, (1973)). The 5-HT2A receptor subtype (also referred to as subclass) is widely yet discretely expressed in the human brain, including many cortical, limbic, and forebrain regions postulated to be involved in the modulation of higher cognitive and affective functions. This receptor subtype is also expressed on mature platelets where it mediates, in part, platelet aggregation, one of the initial steps in the process of vascular thrombosis.

Given the broad distribution of serotonin within the body, it is understandable that tremendous interest in drugs that affect serotonergic systems exists (Gershon, et al., The Peripheral Actions of 5-Hydroxytryptamine, 246 (1989); Saxena, et al., J. Cardiovascular Pharmacol. 15; Supp. 7 (1990)). Serotonin receptors are members of a large human gene family of membrane-spanning proteins that function as transducers of intercellular communication. They exist on the surface of various cell types, including neurons and platelets, where, upon their activation by either their endogenous ligand serotonin or exogenously administered drugs, they change their conformational structure and subsequently interact with downstream mediators of cellular signaling. Many of these receptors, including the 5-HT2A subclass, are G-protein coupled receptors (GPCRs) that signal by activating guanine nucleotide binding proteins (G-proteins), resulting in the generation, or inhibition of, second messenger molecules such as cyclic AMP, inositol phosphates, and diacylglycerol. These second messengers then modulate the function of a variety of intracellular enzymes, including kinases and ion channels, which ultimately affect cellular excitability and function.

At least 15 genetically distinct 5-HT receptor subtypes have been identified and assigned to one of seven families (5-HT1-7). Each subtype displays a unique distribution, preference for various ligands, and functional correlate(s).

Serotonin may be an important component in various types of pathological conditions such as certain psychiatric disorders (depression, aggressiveness, panic attacks, obsessive compulsive disorders, psychosis, schizophrenia, suicidal tendency), certain neurodegencrative disorders (Alzheimer-type dementia, Parkinsonism, Huntington's chorea), anorexia, bulimia, disorders associated with alcoholism, cerebral vascular accidents, and migraine (Meltzer, Neuropsychopharmacology, 21:106 S-115S (1999); Barnes & Sharp, Neuropharmacology, 38:1083-1152 (1999); Glennon, Neurosci. Biobehavioral Rev., 14:35 (1990)). Recent evidence strongly implicates the 5-HT2 receptor subtype in the etiology of such medical conditions as hypertension, thrombosis, migraine, vasospasm, ischemia, depression, anxiety, psychosis, schizophrenia, sleep disorders and appetite disorders.

Schizophrenia is a particularly devastating neuropsychiatric disorder that affects approximately 1% of the human population. It has been estimated that the total financial cost for the diagnosis, treatment, and lost societal productivity of individuals affected by this disease exceeds 2% of the gross national product (GNP) of the United States. Current treatment primarily involves pharmacotherapy with a class of drugs known as antipsychotics. Antipsychotics are effective in ameliorating positive symptoms (e.g., hallucinations and delusions), yet they frequently do not improve negative symptoms (e.g., social and emotional withdrawal, apathy, and poverty of speech).

Currently, nine major classes of antipsychotics are prescribed to treat psychotic symptoms. Use of these compounds is limited, however, by their side effect profiles. Nearly all of the “typical” or older generation compounds have significant adverse effects on human motor function. These “extrapyramidal” side effects, so termed due to their effects on modulatory human motor systems, can be both acute (e.g., dystonic reactions, a potentially life threatening but rare neuroleptic malignant syndrome) and chronic (e.g., akathisias, tremors, and tardive dyskinesia). Drug development efforts have, therefore, focused on newer “atypical” agents free of some of these adverse effects. However, atypical agents also have the potential for serious side effects including increased risk of stroke, abnormal shifts in sleep patterns, extreme tiredness and weakness, metabolic disorders (including hyperglycemia and diabetes), and weight gain. One of the most common reasons for noncompliance and discontinued use of antipsychotic medication is weight gain. Non-compliance can lead to increased hospitalization and health care costs.

Antipsychotic drugs have been shown to interact with a large number of central monoaminergic neurotransmitter receptors, including dopaminergic, serotonergic, adrenergic, muscarinic, and histaminergic receptors. It is likely that the therapeutic and adverse effects of these drugs are mediated by distinct receptor subtypes. The high degree of genetic and pharmacological homology between these receptor subtypes has hampered the development of subtype-selective compounds, as well as the determination of the normal physiologic or pathophysiologic role of any particular receptor subtype. Thus there is a need to develop drugs that are selective for individual receptor classes and subclasses amongst monoaminergic neurotransmitter receptors.

The prevailing theory for the mechanism of action of antipsychotic drugs involves antagonism of dopamine D2 receptors. Unfortunately, it is likely that antagonism of dopamine D2 receptors also mediates the extrapyramidal side effects. Antagonism of 5-HT2A is an alternate molecular mechanism for drugs with antipsychotic efficacy, possibly through antagonism of heightened or exaggerated signal transduction through serotonergic systems. 5-HT2A antagonists are therefore good candidates for treating psychosis without extrapyramidal side effects.

Traditionally, these receptors have been assumed to exist in a quiescent state unless activated by the binding of an agonist (a drug that activates a receptor). It is now appreciated that many, if not most, of the GPCR monoamine receptors, including serotonin receptors, can exist in a partially activated state in the absence of their endogenous agonists. This increased basal activity (constitutive activity) can be inhibited by compounds called inverse agonists. Both agonists and inverse agonists possess intrinsic activity at a receptor, in that they alone can activate or inactivate these molecules, respectively. In contrast, classic or neutral antagonists compete against agonists and inverse agonists for access to the receptor, but do not possess the intrinsic ability to inhibit elevated basal or constitutive receptor responses.

### SUMMARY

Embodiments disclosed herein relate to an isolated form of a compound selected from Formula (I), Formula (II), Formula (III), Formula (IV) and Formula (V), or a pharmaceutically acceptable salt, prodrug, hydrate, solvate, polymorph, or ester thereof.

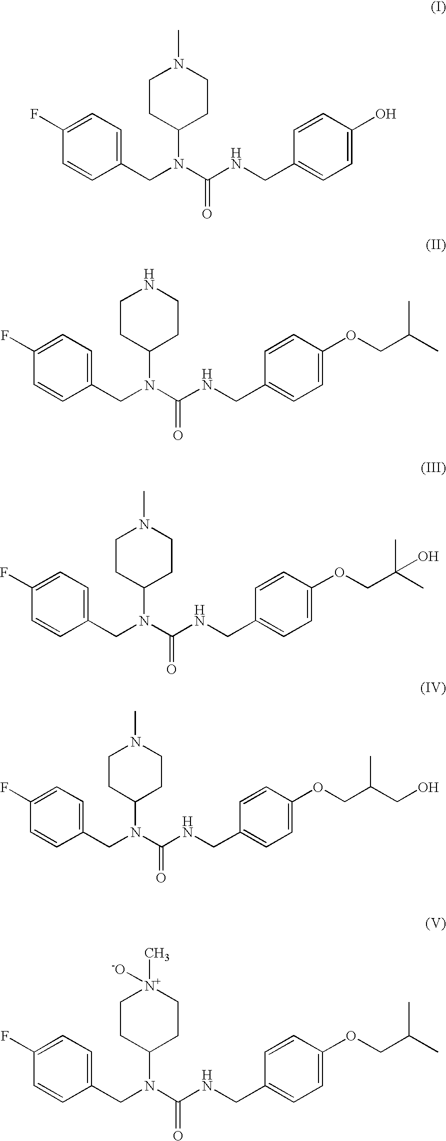

An embodiment disclosed herein relates to a pharmaceutical composition, comprising a therapeutically effective amount of a compound described herein (e.g., an isolated form of a compound of Formulae (I), (II), (III), (IV) and/or (V) as described herein) and a pharmaceutically acceptable carrier, diluent, excipient or combination thereof.

Embodiments disclosed herein relate to a method of inhibiting the activity of a serotonin receptor that can include contacting the monoamine receptor or a system containing a monoamine receptor with at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein).

One embodiment disclosed herein relates to a method of inhibiting an activation of a serotonin receptor that can include contacting the monoamine receptor or a system containing a monoamine receptor with at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein).

Embodiments disclosed herein relate to a method of alleviating or treating one or more disease condition associated with a serotonin receptor that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein).

Embodiments disclosed herein relate to a method of alleviating or treating one or more disease condition associated with a serotonin receptor that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) with the administration of one or more additional therapeutic agents.

Some embodiments disclosed herein relate to a method of alleviating or treating a condition induced by the administration of an anti-psychotic compound that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject being administered the anti-psychotic compound.

An embodiment disclosed herein relates to a method for alleviating or treating a condition associated with dopaminergic therapy that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject receiving dopaminergic therapy.

Embodiments disclosed herein relate to a method of alleviating or treating schizophrenia that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject suffering from schizophrenia.

An embodiment disclosed herein relates to a method of alleviating or treating migraine that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject who suffers from a migraine.

Some embodiments disclosed herein relate to a method of alleviating or treating psychosis that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject suffering from psychosis.

Embodiments disclosed herein relate to a method of alleviating or treating a condition amenable for treatment with an antipsychotic that can include administering a first amount of at least one isolated form of a compound described herein (e.g., an isolated form of a compound of Formulae (I), (II), (III), (IV) and/or (V) as described herein) or a pharmaceutical composition that includes a first amount of at least one isolated form of the compound described herein, and a second amount of an anti-psychotic compound to a subject, wherein the second amount of the anti-psychotic compound is less than the amount of the anti-psychotic compound needed to produce a comparable efficacious effect when the anti-psychotic compound is administered alone.

Some embodiments disclosed herein relate to a method of alleviating or treating a pituitary tumor that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject with a pituitary tumor. In an embodiment, the tumor can be a prolactinoma.

An embodiment disclosed herein relates to a method of inhibiting the formation of a pituitary tumor that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject at risk for forming a pituitary tumor.

Embodiments disclosed herein relate to a method of reducing the level of prolactin in a subject that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject with elevated levels of prolactin.

An embodiment disclosed herein relates to a method of reducing or inhibiting weight gain that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject at risk of gaining weight.

Embodiments disclosed herein relate to a method of alleviating or treating a sleep disorder that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject suffering from a sleep disorder.

Some embodiments disclosed herein relate to a method of increasing slow-wave sleep that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject.

An embodiments disclosed herein relates to a method of alleviating or treating insomnia that can include administering a sleep-inducing agent adapted to induce onset of sleep in a subject; and administering to the subject a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to maintain the sleep induced by the sleep-inducing agent.

Embodiments disclosed herein relate to a method of alleviating or treating sleep maintenance insomnia that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject suffering from sleep maintenance insomnia at a frequency of every other day or greater.

An embodiment disclosed herein relates to a method for identifying a compound which binds to a serotonin receptor that can include labeling an isolated form of a compound described herein; with a detectable label; contacting the serotonin receptor with the labeled compound; and determining whether the labeled compound binds to the serotonin receptor. In an embodiment, the detectable label can be a radiolabel such as [³H], [¹⁸F], [¹¹C] and [¹²⁵I].

### DETAILED DESCRIPTION

Embodiments disclosed herein relate to an isolated form of a compound selected from Formula (I), Formula (II), Formula (III), Formula (IV) and Formula (V), or a pharmaceutically acceptable salt, prodrug, hydrate, solvate, polymorph, or ester thereof. In an embodiment, the isolated form of a compound selected from Formulae (I), (II), (III), (IV) and (V) can include at least 75% of the compound. In another embodiment, the isolated form of a compound selected from Formulae (I), (II), (III), (IV) and (V) can include at least 80% of the compound. In yet another embodiment, the isolated form of a compound selected from Formulae (I), (II), (III), (IV) and (V) can include at least 85% of the compound. In yet still another embodiment, the isolated form of a compound selected from Formulae (I), (II), (III), (IV) and (V) can include at least 90% of the compound. In another embodiment, the isolated form of a compound selected from Formulae (I), (II), (III), (IV) and (V) can include at least 95% of the compound. In another embodiment, the isolated form of a compound selected Formulae (I), (II), (III), (IV) and (V) can include at least 99% of the compound. Compounds of Formulae (I), (II), (III), (IV) and (V) can be produced synthetically and have been shown to be metabolites of N-(1-methylpiperidin-4-yl)-N-(4-fluorophenylmethyl)-N′-(4-(2-methylpropyloxy)phenylmethyl) carbamide.

Unless defined otherwise, all technical and scientific terms used herein have the same meaning as is commonly understood by one of ordinary skill in the art. All patents, applications, published applications and other publications referenced herein are incorporated by reference in their entirety. In the event that there are a plurality of definitions for a term herein, those in this section prevail unless stated otherwise.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure or be stereoisomeric mixtures. In addition it is understood that, in any compound described herein having one or more double bond(s) generating geometrical isomers that can be defined as E or Z each double bond may independently be E or Z a mixture thereof. Likewise, all tautomeric forms are also intended to be included.

As used herein, “pharmaceutically acceptable salf” refers to a salt of a compound that does not abrogate the biological activity and properties of the compound. Pharmaceutical salts can be obtained by reaction of a compound disclosed herein with an acid or base. Base-formed salts include, without limitation, ammonium salt (NH₄⁺); alkali metal, such as, without limitation, sodium or potassium, salts; alkaline earth, such as, without limitation, calcium or magnesium, salts; salts of organic bases such as, without limitation, dicyclohexylamine, N-methyl-D-glucamine, tris(hydroxymethyl)methylamine; and salts with the amino group of amino acids such as, without limitation, arginine and lysine. Useful acid-based salts include, without limitation, hydrochlorides, hydrobromides, sulfates, nitrates, phosphates, methanesulfonates, ethanesulfonates, p-toluenesulfonates and salicylates.

An “agonist” is defined as a compound that increases the basal activity of a receptor (i.e. signal transduction mediated by the receptor).

As used herein, “partial agonist” refers to a compound that has an affinity for a receptor but, unlike an agonist, when bound to the receptor it elicits only a fractional degree of the pharmacological response normally associated with the receptor even if a large number of receptors are occupied by the compound.

An “inverse agonist” is defined as a compound that decreases the basal activity of a receptor (i.e., signaling mediated by the receptor). Such compounds are also known as negative antagonists. An inverse agonist is a ligand for a receptor that causes the receptor to adopt an inactive state relative to a basal state occurring in the absence of any ligand. Thus, while an antagonist can inhibit the activity of an agonist, an inverse agonist is a ligand that can alter the conformation of the receptor in the absence of an agonist. The concept of an inverse agonist has been explored by Bond et al. in Nature 374:272 (1995). More specifically, Bond et al. have proposed that ligand free β₂-adrenoceptor exists in equilibrium between an inactive conformation and a spontaneously active conformation. Agonists are proposed to stabilize the receptor in an active conformation. Conversely, inverse agonists are believed to stabilize an inactive receptor conformation. Thus, while an antagonist manifests its activity by virtue of inhibiting an agonist, an inverse agonist can additionally manifest its activity in the absence of an agonist by inhibiting the spontaneous conversion of an unliganded receptor to an active conformation.

As used herein, “antagonist” refers to a compound that competes with an agonist or inverse agonist for binding to a receptor, thereby blocking the action of an agonist or inverse agonist on the receptor. An antagonist attenuates the action of an agonist on a receptor. However, an antagonist (also known as a “neutral agonist”) has no effect on constitutive receptor activity. An antagonist may bind reversibly or irreversibly, and may reduce the activity of the receptor until the antagonist is metabolized or dissociates or is otherwise removed by a physical or biological process.

As used herein, “IC₅₀” refers to an amount, concentration, or dosage of a particular test compound that achieves a 50% inhibition of a maximal response. The IC₅₀ can be determined using by an assay. The assay may be an R-SAT® assay as described herein but is not limited to an RSAT assay.

As used herein, “EC₅₀” refers to an amount, concentration or dosage of a particular test compound that elicits a dose-dependent response at 50% of maximal expression of a particular response that is induced, provoked or potentiated by the particular test compound, in an assay that measures such response such as but not limited to R-SAT® assay described herein.

As used herein, a “subject” refers to an animal that is the object of treatment, observation or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and, in particular, humans.

As used herein, a “patient” refers to a subject that is being treated in order to attempt to cure, or at least ameliorate the effects of, a particular disease or disorder or to prevent the disease or disorder from occurring in the first place.

As used herein, the terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy. Furthermore, treatment may include acts that may worsen the patient's overall feeling of well-being or appearance.

The term “therapeutically effective amount” is used to indicate an amount of an active compound, or pharmaceutical agent, that elicits the biological or medicinal response indicated. For example, a therapeutically effective amount of compound can be the amount need to prevent, alleviate or ameliorate symptoms of disease or prolong the survival of the subject being treated This response may occur in a tissue, system, animal or human and includes alleviation of the symptoms of the disease being treated. Determination of a therapeutically effective amount is well within the capability of those skilled in the art, especially in light of the detailed disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” refers to an inert substance that is added to a pharmaceutical composition to provide, without limitation, bulk, consistency, stability, binding ability, lubrication, disintegrating ability etc., to the composition. A “diluent” is a type of excipient.

The terms “pure,” “purified,” “substantially purified,” and “isolated” as used herein refer to the compound of the embodiment being free of other, dissimilar compounds with which the compound, if found in its natural state, would be associated in its natural state. In some embodiments described as “pure,” “purified,” “substantially purified,” or “isolated” herein, the compound may comprise at least 75%, 80%, 85%, 90%, 95%, 99% of the mass, by weight, of a given sample.

### Synthesis

Compounds of Formulae (I), (II), (III), (IV) and (V) as described herein may be prepared in various ways. General synthetic routes to the compounds of Formulae (I), (II), (III), (IV) and (V) are shown in Schemes A-E. The routes shown are illustrative only and are not intended, nor are they to be construed, to limit the scope of this invention in any manner whatsoever. Those skilled in the art will be able to recognize modifications of the disclosed synthesis and to devise alternate routes based on the disclosures herein; all such modifications and alternate routes are within the scope of this application.

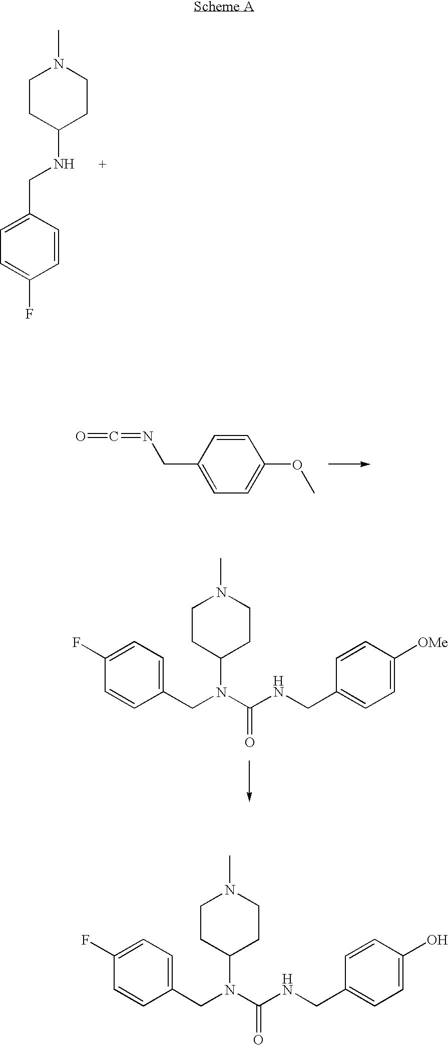

Scheme A shows a general reaction scheme for forming the compound of Formula (I). As shown in Scheme A, the secondary amine and isocyanate can be combined to produce the 4-methoxybenzyl derivative of the compound of Formula (I). The methoxy group can be converted to a hydroxy group using methods known to those skilled in the art, for example, using a boron trihalide to form the compound of Formula (I).

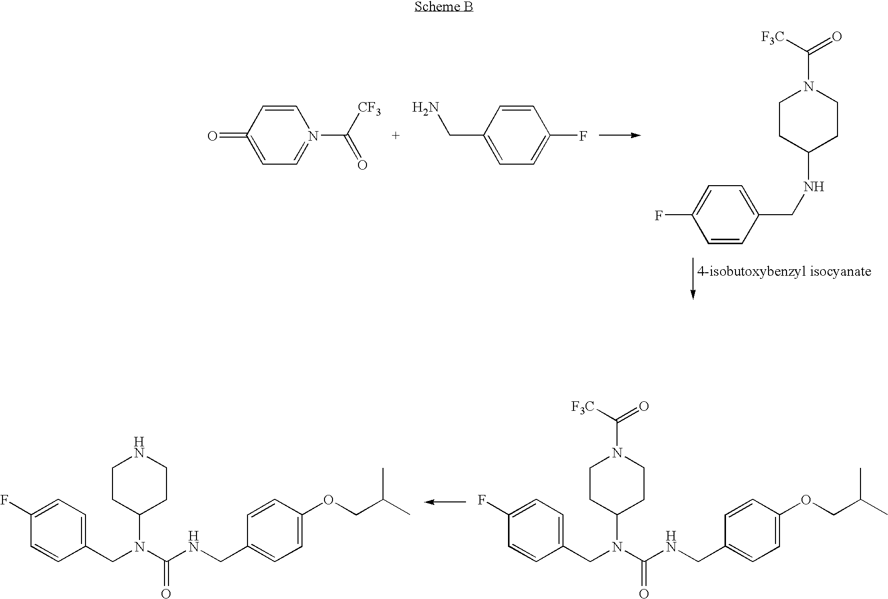

An exemplary method for synthesizing the compound of Formula (II) is shown in Scheme B. The protected 4-piperidoinone and 4-fluorobenzylamine can undergo reductive amination to form N-(4-fluorobenzyl)-4-amino-1-trifluoroacetylpiperidine. The resulting secondary amine can then be reacted with the appropriate isocyanate to form the nitrogen-protected carbamide. The acyl protecting group can be cleaved off using an alkali metal salt such as potassium carbonate to form the compound of Formula (II).

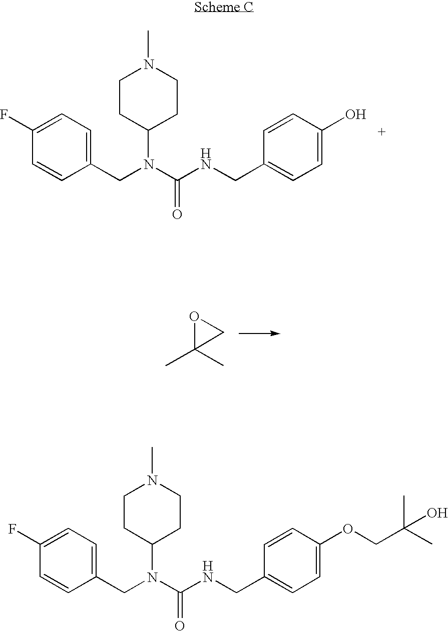

One method for synthesizing the compound of Formula (III) is shown in Scheme C. The compound of Formula (I) can be reacted with isobutylene oxide to form the compound of Formula (III) via a nucleophilic ring opening of the epoxide.

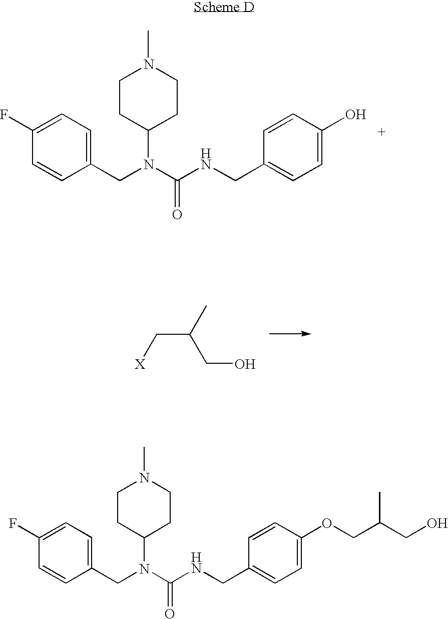

Scheme D shows a general reaction scheme for forming the compound of Formula (IV). As shown in Scheme D, the compound of Formula (I) can be reacted with a halohydrin to form the compound of Formula (IV). All the compounds described herein can be purified using methods known to those skilled in art.

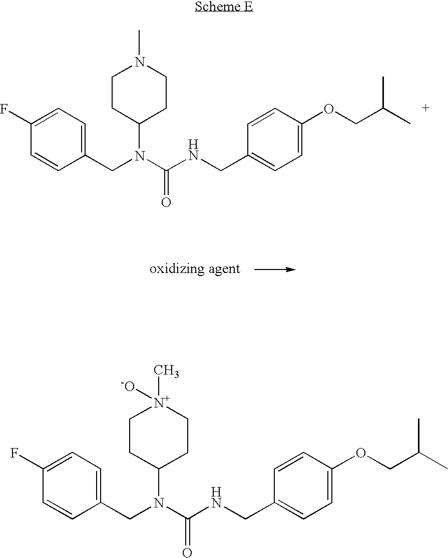

One example of a method for synthesizing a compound of Formula (V) is shown in Scheme E. As shown in Scheme E, N-(1-methylpiperidin-4-yl)-N-(4-fluorophenylmethyl)-N′-(4-(2-methylpropyloxy)phenylmethyl) carbamide can be oxidized with a suitable oxidizing agent to form a compound of Formula (V). Suitable oxidizing agents are known to those skilled in the art. One example of a suitable oxidizing agent is meta-chloroperbenzoic acid. All the compounds described herein can be purified using methods known to those skilled in art.

### Pharmaceutical Compositions

An embodiment disclosed herein relates to a pharmaceutical composition, comprising a therapeutically effective amount of a compound described herein (e.g., an isolated form of a compound of Formulae (I), (II), (III), (IV) and/or (V) as described herein) and a pharmaceutically acceptable carrier, diluent, excipient or combination thereof. In some embodiments, the pharmaceutical composition that includes a therapeutically effective amount of an isolated form of a compound of Formulae (I), (II), (III), (IV) and/or (V) can also include one or more additional therapeutic agents.

Suitable additional therapeutic agents include, but are not limited to, dopaminergic agents, anti-dyskensia agents, anti-dystonia agents, anti-myoclonus agents, anti-tremor agents, anti-psychotic agents, antidepressants, anti-dementia agents and sleep-inducing agents. In an embodiment, the dopaminergic agent can be selected from levodopa (such as SINEMET™, SINEMET-CR™), bromocriptine (such as PARLODEL™), pergolide (such as PERMAX™), ephenedrine sulfate (such as EPHEDRINE™), pemoline such as CYLERT™), mazindol (such as SANOREX™), d,1-α-methylphenethylamine (such as ADDERALL™), methylphenydate (such as RITALIN™), pramipexole (such as MIRAPEX™), modafinil (such as PROVIGIL™), and ropinirole (such as REQUIP™).

In some embodiments, the anti-dyskensia agent, anti-dystonia, anti-myoclonus, or anti-tremor agent can be selected from baclofen (such as LIORESAL™) botulinum toxin (such as BOTOX™), clonazepam (such as KLONOPIN™), and diazepam (such as VALIUM™).

In an embodiment, the anti-psychotic agent can be selected from chlorpromazine (such as THORAZINE™), haloperidol (such as HALDOL™), molindone (such as MOBAN™), thioridazine (such as MELLARIL™), a phenothiazine, a butyrophenome, a phenylbutylpiperadine, thioxanthine (such as fluphenthixol), a substituted benzamide (such as sulpiride), sertindole, amisulpride, risperidone, clozapine, olanzapine, ziprasidone, a debenzapine, a benzisoxidil, a salt of lithium, Aripiprazole (such as Abilify®), Etrafon®, Droperidol (such as Inapsine®), Thioridazine (such as Mellaril®), Thiothixene (such as Navane®), Promethazine (such as Phenergan®), Metoclopramide (such as Reglan®), Chlorprothixene (such as Taractan®), Triavil®, Molindone (such as Moban®), Sertindole (such as Serlect®), Droperidol, Amisulpride (such as Solian®), Melperone, Paliperidone (such as Invega®), Tetrabenazine and their active metabolites. Exemplary phenothiazines include chlorpromazine (such as Thorazine®), mesoridazine (such as Serentil®), prochlorperazine (such as Compazine®), thioridazine (such as Mellaril), Fluphenazine (such as Prolixin®), Perpehnazine (such as Trilafon®), and Trifluoperazine (such as Stelazine®). An example of a suitable phenylbutylpiperadine is pimozide (such as Orap®). A non-limiting list of debenzapines include clozapine (such as Clozaril®), loxapine (such as Loxitane®), olanzapine (such as Zyprexa®), and quetiapine (such as Seroquel®). A representative benzisoxidil is ziprasidone (such as Geodon®). An example of a lithium salt is lithium carbonate.

In an embodiment, the antidepressant can be selected from citalopram, escitalopram oxalate, fluoxetine, fluvoxamine maleate, paroxetine, sertraline, and dapoxetine.

In one embodiment, the anti-dementia agent can be a cholinesterase inhibitor such as donepezil (such as Aricept), galantamine (such as Razadyne) rivastigmine (such as Exelon), tacrine, metrifonate, physostigmine, neostigmine, pyridostigmine, ambenonium, demarcarium, aldicarb, bendiocarb, bufencarb, carbaryl, carbendazim, carbetamide, carbofuran, chlorbufam, chloropropham, ethiofencarb, formetanate, methiocarb, methomyl, oxamyl, phenmedipham, pinmicarb, pirimicarb, propamocarb, propham, propoxur, edrophonium, phenothiazines, echothiophate, diisopropyl fluorophosphate, dimebon, Huperzine A, T-82 ((2-[2-(1-benzylpiperidin-4-yl)ethyl]-2,3-dihydro-9-methoxy-1H-pyrrolo[3,4-b]quinolin-1-one hemifumarate)), TAK-147 (zanapezil), phenserine, quilostigmine, ganstigmine, butyrophenones, imipramines, tropates, phencyclidines, curariforms, ethephon, ethopropazine, iso-OMPA, tetrahydrofurobenzofuran cymserine, N¹phenethyl-norcymserine, N⁸-benzylnorcymserine, N¹,N⁸-bisnorcymserine, N¹-N⁸-bisbenzylnorphysostigmine, N¹, N⁸-bisbenzylnorphenserine and N¹, N⁸-bisbenzylnorcymserine.

In some embodiments, the sleep-inducing agent can be selected from zolpidem, eszopiclone, a benzodiazepine, a melatonin agonist, and an antihistamine. A non-limiting list of benzodiazepines include temazepam, diazepam, lorazepam, nitrazepam, and midazolam. An exemplary melatonin agonist is ramelteon. An example of a suitable antihistamine is diphenhydramine.

The term “pharmaceutical composition” refers to a mixture of a compound disclosed herein with other chemical components, such as diluents or carriers. The pharmaceutical composition facilitates administration of the compound to an organism. Multiple techniques of administering a compound exist in the art including, but not limited to, oral, intramuscular, intraocular, intranasal, intravenous, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like. Pharmaceutical compositions will generally be tailored to the specific intended route of administration.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990, which is hereby incorporated by reference in its entirety.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, intraocular injections or as an aerosol inhalant.

Alternatively, one may administer the compound in a local rather than systemic manner, for example, via injection of the compound directly into the area of pain or inflammation, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes.

Pharmaceutical compositions for use in accordance with the present disclosure thus may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., as disclosed in Remington's Pharmaceutical Sciences, cited above.

For injection, the agents disclosed herein may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds disclosed herein to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination disclosed herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations, which can be used orally, include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present disclosure are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances, which increase the viscosity of the suspension, such as sodium carboxymethyl cellulose, sorbitol, or dextran. Optionally, the suspension may also contain suitable stabilizers or agents, which increase the solubility of the compounds to allow for the preparation of highly, concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

An exemplary pharmaceutical carrier for the hydrophobic compounds disclosed herein is a co-solvent system comprising benzyl alcohol, a nonpolar surfactant, a water-miscible organic polymer, and an aqueous phase. A common co-solvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of Polysorbate 80™; the fraction size of polyethylene glycol may be varied; and other biocompatible polymers may replace polyethylene glycol, e.g., polyvinyl pyrrolidone. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Many of the compounds used in the pharmaceutical combinations disclosed herein may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acids or base forms.

The exact formulation, route of administration and dosage for the pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Typically, the dose range of the composition administered to the patient can be from about 0.5 to 1000 mg/kg of the patient's body weight, or 1 to 500 mg/kg, or 10 to 500 mg/kg, or 50 to 100 mg/kg of the patient's body weight. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

Although the exact dosage will be determined on a drug-by-drug basis, in most cases, some generalizations regarding the dosage can be made. The daily dosage regimen for an adult human patient may be, for example, an oral dose of between 0.1 mg and 500 mg of each ingredient, preferably between 1 mg and 250 mg, e.g. 5 to 200 mg or an intravenous, subcutaneous, or intramuscular dose of each ingredient between 0.01 mg and 100 mg, preferably between 0.1 mg and 60 mg, e.g. 1 to 40 mg of each ingredient of the pharmaceutical compositions disclosed herein or a pharmaceutically acceptable salt thereof calculated as the free base, the composition being administered 1 to 4 times per day. Alternatively the compositions disclosed herein may be administered by continuous intravenous infusion, preferably at a dose of each ingredient up to 400 mg per day. Thus, the total daily dosage by oral administration of each ingredient will typically be in the range 1 to 2000 mg and the total daily dosage by parenteral administration will typically be in the range 0.1 to 400 mg. In some embodiments, the compounds will be administered for a period of continuous therapy, for example for a week or more, or for months or years.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen, which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device, which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound disclosed herein formulated in a compatible pharmaceutical carrier may also be prepared, placed in an appropriate container, and labeled for treatment of an indicated condition.

### Methods of Use

Embodiments disclosed herein relate to a method of inhibiting the activity of a serotonin receptor that can include contacting the monoamine receptor or a system containing a monoamine receptor with at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein). In an embodiment the activity can be a signaling activity. In some embodiments, the activity can be constitutive. In one embodiment, the activity can be associated with serotonin receptor activation.

One embodiment disclosed herein relates to a method of inhibiting an activation of a serotonin receptor that can include contacting the monoamine receptor or a system containing a monoamine receptor with at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein). In some embodiment, the activity can be by an agonist agent. In an embodiment, the agonistic agent can be exogenous. In an embodiment, the agonistic agent can be endogenous. In some embodiment, the activation can be constitutive.

Embodiments disclosed herein relate to a method of alleviating or treating one or more disease condition associated with a serotonin receptor that can include administering a therapeutically effective amount of at least one isolated form of a compound described herein (e.g., an isolated form of a compound of Formulae (I), (II), (III), (IV) and/or (V) as described herein) or a pharmaceutical composition described herein, such as a pharmaceutical composition that includes a therapeutically effective amount of at least one isolated form of a compound of Formulae (I), (II), (III), (IV) and/or (V). In an embodiment) the disease condition can be a neuropsychiatric disorder. Exemplary neuropsychiatric disorder include, but are not limited to, schizophrenia, schizoaffective disorder, mania, depression, a cognitive disorder, aggressiveness, panic attacks, obsessive compulsive disorder, borderline personality disorder, borderline disorder, multiplex developmental disorder (MDD), a behavioral disorder, psychosis, suicidal tendency, bipolar disorder, sleep disorder, addiction, attention deficit hyperactivity disorder (ADHD), post traumatic stress disorder (PTSD), Tourette's syndrome, anxiety, autism, Down's syndrome, a learning disorder, a psychosomatic disorder, alcohol withdrawal, epilepsy, pain, a disorder associated with hypoglutamatergia, and/or serotonin syndrome. In an embodiment, the depression can be dysthymia, SSRI-resistant depression and/or depression associated with psychosis. In an embodiment, the aggressiveness can be impulsive aggression. In one embodiment, the behavioral disorder can be associated with age-related dementia. When the disease condition is psychosis, the psychosis can be caused or results from various different origins. For example, the psychosis can be the result of drugs, treatment, and/or disease. Exemplary diseases that can cause psychosis include dementia, post traumatic stress disorder, Alzheimer's disease, and schizophrenia. In an embodiment, the psychosis can be Parkinson's disease psychosis. In an embodiment, the psychosis can be Alzheimer's disease-induced psychosis. In an embodiment, the psychosis can be dementia-related psychosis. In an embodiment, the psychosis can be the result of schizophrenia. In some embodiments, the sleep disorder can be selected from sleep maintenance insomnia, chronic insomnia, transient insomnia and periodic limb movements during sleep (PLMS). In an embodiment, the addiction can be selected from drug addiction, alcohol addiction, opioid addiction and nicotine addiction. In one embodiment, the anxiety can be general anxiety disorder (GAD). In some embodiments, the pain can be selected from chronic pain, neuropathic pain, inflammatory pain, diabetic peripheral neuropathy, fibromyalgia, postherpetic neuralgia and reflex sympathetic dystrophy. In an embodiment, the disease condition can be a cognitive disorder.

In another embodiment, the disease condition can be a neurodegenerative disorder. Examples of neurodegenerative disorders are Alzheimer's disease, Parkinson's disease, Huntington's chorea, sphinocerebellar atrophy, frontotemporal dementia, supranuclear palsy and Lewy body dementia.

In some embodiments, the disease condition can be chemotherapy-induced emesis, frailty, on/off phenomena, non-insulin-dependent diabetes mellitus, metabolic syndrome, an autoimmune disorder, sepsis, increased intraocular pressure, glaucoma, a retinal disease, Charles Bonnet syndrome, substance abuse, sleep apnea, pancreatis, anorexia, bulimia, a disorder associated with alcoholism, a cerebral vascular accident, amyotrophic lateral sclerosis, AIDS related dementia, traumatic brain, traumatic spinal injury, tinnitus, a menopausal symptom, sexual dysfunction, low male fertility, low sperm motility, hair loss, hair thinning, incontinence, hemorrhoids, migraine, hypertension, thrombosis, abnormal hormonal activity, a hormonal disorder, a pituitary tumor, a side effect associated with a pituitary tumor, vasospasm, ischemia, cardiac arrhythmia, cardiac insufficiency, asthma, emphysema, and/or an appetite disorder. In one embodiment, the autoimmune disorders can be lupus or multiple sclerosis. In an embodiment, the retinal disease can be age related macular degeneration. In some embodiments, the menopausal symptom can be hot flashes. In an embodiment, the sexual dysfunction can be selected from female sexual dysfunction, female sexual arousal dysfunction, hypoactive sexual desire disorder, decreased libido, pain, aversion, female orgasmic disorder and an ejaculatory problem. In an embodiment, the thrombosis can be associated with myocardial infarction, stroke, idiopathic thrombocytopenic purpura, thrombotic thrombocytopenic purpura, and/or peripheral vascular disease. In some embodiments, the abnormal hormonal activity can be abnormal levels of ACTH, corticosterone, rennin, and/or prolactin. In one embodiment, the hormonal disorder can be Cushing's disease, Addison's disease, and/or hyperprolactinemia. In some embodiments, the side effect associated with a pituitary tumor can be selected from hyperprolactinemia, infertility, changes in menstruation, amenorrhea, galactorrhea, loss of libido, vaginal dryness, osteoporosis, impotence, headache, blindness and double vision.

In some embodiments, the disease condition can be associated with dysfunction of the serotonin receptor, activation of the serotonin receptor and/or increased activity of the serotonin receptor. With respect to the serotonin receptor, in some embodiments, the serotonin receptor can be a 5-HT2A subclass serotonin receptor. In an embodiment, the serotonin receptor can be a 5-HT2C subclass serotonin receptor. The location of the serotonin receptor can vary. For example, the serotonin receptor can be in the central nervous system, the peripheral nervous system and/or in blood cells or platelets. In an embodiment, the serotonin receptor can be mutated or modified.

Embodiments disclosed herein relate to a method of alleviating or treating one or more disease condition associated with a serotonin receptor that can include administering a therapeutically effective amount of at least one isolated form of a compound described herein or a pharmaceutical composition described herein with the administration of one or more additional therapeutic agents.

Exemplary additional therapeutic agents include, but are not limited to, dopaminergic agents, anti-dyskensia agents, anti-dystonia agents, anti-myoclonus agents, anti-tremor agents, anti-psychotic agents, antidepressants, anti-dementia agents and sleep-inducing agents. In an embodiment, the dopaminergic agent can be selected from levodopa (such as SINEMET™, SINEMET-CR™, bromocriptine (such as PARLODEL™), pergolide (such as PERMAX™), ephenedrine sulfate (such as EPHEDRINE™), pemoline such as CYLERT™), mazindol (such as SANOREX™), d,1-α-methylphenethylamine (such as ADDERALL™), methylphenydate (such as RITALIN™), pramipexole (such as MIRAPEX™), modafinil (such as PROVIGIL™), and ropinirole (such as REQUIP™).

In some embodiments, the anti-dyskensia agent, anti-dystonia, anti-myoclonus, or anti-tremor agent can be selected from baclofen (such as LIORESAL™, botulinum toxin (such as BOTOX™), clonazepam (such as KLONOPIN™), and diazepam (such as VALIUM™).

In an embodiment, the anti-psychotic agent can be selected from chlorpromazine (such as THORAZINE™), haloperidol (such as HALDOL™), molindone (such as MOBAN™), thioridazine (such as MELLARIL™), a phenothiazine, a butyrophenome, a phenylbutylpiperadine, thioxanthine (such as fluphenthixol), a substituted benzamide (such as sulpiride), sertindole, amisulpride, risperidone, clozapine, olanzapine, ziprasidone, a debenzapine, a benzisoxidil, a salt of lithium, Aripiprazole (such as Abilify®), Etrafon®, properidol (such as Inapsine®), Thioridazine (such as Mellaril®), Thiothixene (such as Navane®), Promethazine (such as Phenergan®), Metoclopramide (such as Reglan®), Chlorprothixene (such as Taractan®), Triavil®, Molindone (such as Moban®), Sertindole (such as Serlect®), Amisulpride (such as Solian®), Melperone, Paliperidone (such as Invega®), Tetrabenazine and their active metabolites. Exemplary phenothiazines include chlorpromazine (such as Thorazine®), mesoridazine (such as Serentil®), prochlorperazine (such as Compazine®), thioridazine (such as Mellaril), Fluphenazine (such as Prolixin®), Perpehnazine (such as Trilafon®), and Trifluoperazine (such as Stelazine®). Ah example of a suitable phenylbutylpiperadine is pimozide (such as Orap®). A non-limiting list of debenzapines include clozapine (such as Clozaril®), loxapine (such as Loxitane®), olanzapine (such as Zyprexa®), and quetiapine (such as Seroquel®). A representative benzisoxidil is ziprasidone (such as Geodon®). An example of a lithium salt is lithium carbonate.

In an embodiment, the antidepressant can be selected from citalopram, escitalopram oxalate, fluoxetine, fluvoxamine maleate, paroxetine, sertraline, and dapoxetine.

In one embodiment, the anti-dementia agent can be a cholinesterase inhibitor such as donepezil (such as Aricept), galantamine (such as Razadyne) rivastigmine (such as Exelon), tacrine, metrifonate, physostigmine, neostigmine, pyridostigmine, ambenonium, demarearium, aldicarb, bendiocarb, bufencarb, carbaryl, carbendazim, carbetamide, carbofuran, chlorbufam, chloropropham, ethiofencarb, formetanate, methiocarb, methomyl, oxamyl, phenmedipham, pinmicarb, pirimicarb, propamocarb, propham, propoxur, edrophonium, phenothiazines, echothiophate, diisopropyl fluorophosphate, dimebon, Huperzine A, T-82 ((2-[2-(1-benzylpiperidin-4-yl)ethyl]-2,3-dihydro-9-methoxy-1H-pyrrolo[3,4-b]quinolin-1-one hemifumarate)), TAK-147 (zanapezil), phenserine, quilostigmine, ganstigmine, butyrophenones, imipramines, tropates, phencyclidines, curariforms, ethephon, ethopropazine, iso-OMPA, tetrahydrofurobenzofuran cymserine, N¹phenethyl-norcymserine, N⁸-benzylnorcymserine, N¹,N⁸-bisnorcymserine, N¹-N⁸-bisbenzylnorphysostigmine, N¹,N⁸-bisbenzylnorphenserine and N¹, N⁸-bisbenzylnorcymserine.

In some embodiments, the sleep-inducing agent can be selected from zolpidem, eszopiclone, a benzodiazepine, a melatonin agonist, and an antihistamine. A non-limiting list of benzodiazepines include temazepam, diazepam, lorazepam, nitrazepam, and midazolam. An exemplary melatonin agonist is ramelteon. An example of a suitable antihistamine is diphenhydramine.

Some embodiments disclosed herein relate to a method of alleviating or treating a condition induced by the administration of an anti-psychotic compound that can include administering a therapeutically effective amount of at least one isolated form of a compound described herein or a pharmaceutical composition described herein to a subject being administered the anti-psychotic compound. In some embodiments, the antipsychotic compound can have broad activity at multiple monoamine receptors subtypes. In an embodiment, the antipsychotic compound is a typical antipsychotic. In other embodiments, the antipsychotic compound can be an atypical antipsychotic. In an embodiment, the antipsychotic compound can be a D2 antagonist. In some embodiments, the condition induced by the anti-psychotic compound can be a side effect selected from an extrapyramidal side effect, a histaminic side effect, an alpha adrenergic side effect, and an anticholinergic side effect. Additional conditionals that can be induced by the anti-psychotic compound include stroke, tremors, sedation, gastrointestinal problems, neurological problems, increased risk of death, a cerebrovascular event, a movement disorder, dystonia, akathisia, a parkinsoniam movement disorder, dyskinesia, tardive dyskinesia, a cognitive disorder, prolactinemia, catalepsy, psychosis, neuroleptic malignant syndrome, a heart problem, a pulmonary problem, diabetes, liver failure, suicidality, sedation, orthostatic hypotension, choking, dizziness, tachycardia, blood abnormalities, an abnormal triglyceride level, an increased cholesterol level, dyslipidemia, hyperglycemia, syncope, a seizure, dysphagia, priapism, thrombotic thrombocytopenic purpura, disruption of body temperature regulation, insomnia, agitation, anxiety, somnolence, aggressive reaction, headache, constipation, nausea, dyspepsia, vomiting, abdominal pain, saliva increase, toothache, rhinitis, coughing, sinusitis, pharyngitis, dyspnea, back pain, chest pain, fever, rash, dry skin, seborrhea, increased upper respiratory infection, abnormal vision, arthralgia, hypoaesthesia, manic reaction, concentration impairment, dry mouth, pain, fatigue, acne, pruritus, myalgia, skeletal pain, hypertension, diarrhea, confusion, asthenia, urinary incontinence, sleepiness, increased duration of sleep, accommodation disturbance, palpitations, erectile dysfunction, ejaculatory dysfunction, orgastic dysfunction, lassitude, increased pigmentation, increased appetite, automatism, increased dream activity, diminished sexual desire, nervousness, depression, apathy, catatonic reaction, euphoria, increased libido, amnesia, emotional liability, a nightmare, delirium, yawning, dysarthria, vertigo, stupor, paraesthesia, aphasia, hypoesthesia, tongue paralysis, a leg cramp, torticollis, hypotonia, coma, migrain, hyperreflexia, choreoathetosis, anorexia, flatulence, stomatitis, melena, hemorrhoids, gastritis, fecal incontinence, erutation, gastroeophageal reflux, gastroenteritis, esophagitis, tongue discoloration, choleithiasis, tongue edema, diverticulitis, gingivitis, discolored feces, gastrointestinal hemorrhage, hematemesis, edema, rigors, malaise, pallor, enlarged abdomen, ascites, sarcoidosis, flushing, hyperventilation, bronchospasm, pneumonia, tridor, asthma, increased sputum, aspiration, photosensitivity, increased sweating, acne, decreased sweating, alopecia, hyperkeratosis, skin exfoliation, bullous eruption, skin ulceration, aggravated psoriasis, furunculosis, verruca, dermatitis lichenoid, hypertrichosis, genital pruritus, urticaria, ventricular tachycardia, angina pectoris, premature atrial contractions, T wave inversion, a ventricular extrasystole, ST depression, AV block, myocarditis, abnormal accommodation, xerophthalmia, diplopia, eye pain, blepharitis, photopsia, photophobia, abnormal lacrimation, hyponatremia, creatine phosphokinase increase, thirst, weight decrease, decreased serum iron, cachexia, dehydration, hypokalemia, hypoproteinemia, hyperphosphatemia, hypertrigylceridemia, hyperuricemia, hypoglycemia, polyuria, polydipsia, hemturia, dysuria, urinary retention, cystitis, renal insufficiency, arthrosis, synostosis, bursitis, arthritis, menorrhagia, dry vagina, nonpeurperal lactation, amenorrhea, female breast pain, leukorrhea, mastitis, dysmenorrhea, female perineal pain, intermenstrual bleeding, vaginal hemorrhage, increased SGOT, increased SGPT, cholestatic hepatitis, cholecystitis, choleithiasis, hepatitis, hepatocellular damage, epistaxis, superficial phlebitis, thromboplebitis, thrombocytopenia, tinnitus, hyperacusis, decreased hearing, anemia, hypochromic anemia, normocytic anemia, granulocytopenia, leukocytosis, lymphadenopathy, leucopenia, Pelger-Huet anomaly, gynceomastia, male breast pain, antiduretic hormone disorder, bitter taste, micturition disturbances, oculogyric crisis, abnormal gait, involuntary muscle contraction, increased injury, a pituitary tumor, galactorrhea, bradykinesia, myoclonus, hiccups, uncontrolled gambling, a drug craving, rigidity, psychomotor slowing, tics, Friedrich's ataxia, Machado-Joseph's disease, restless legs syndrome, and a hallucinogenic effect. In an embodiment, the dyskinesia can be induced by treatment of Parkinson's disease. In an embodiment, the akathisia can be induced by administration of a neuroleptic agent or selective serotonin reuptake inhibitor. In some embodiments, the subject who is being administered the anti-psychotic compound is being treated for a disease or disorder selected from schizophrenia, bipolar disorder, agitation, psychosis, behavioral disturbances in Alzheimer's disease, depression with psychotic features or bipolar manifestations, obsessive compulsive disorder, post traumatic stress syndrome, anxiety, personality disorders (borderline and schizotypal), dementia, dementia with agitation, dementia in the elderly, Tourette's syndrome, restless leg syndrome, insomnia, social anxiety disorder, dysthymia, ADHD, and autism.

An embodiment disclosed herein relates to a method for alleviating or treating a condition associated with dopaminergic therapy that can include administering a therapeutically effective amount of at least one isolated form of a compound described herein or a pharmaceutical composition described herein to a subject receiving dopaminergic therapy. In some embodiments, the subject can have a neurodegenerative disease such as Alzheimer disease, Parkinson's disease, Huntington's chorea, sphinocerebellar atrophy, frontotemporal dementia, supranuclear palsy, and/or Lewy body dementia. In an embodiment, the dopaminergic therapy can include the administration of a compound selected from levodopa, (such as SINAMET™, SINAMETCR™), bromocriptine (such as PARLODEL™), pergolide (such as PERMAX™), ephenedrine sulfate (such as EPHEDRINE™), pemoline such as CYLERT™), mazindol (such as SANOREX™), d,1-α-methylphenethylamine (such as ADDERALL™), methylphenydate (such as RITALIN™), pramipexole (such as MIRAPEXT™), modafinil (such as PROVIGIL™), and ropinirole (such as REQUIP™). In some embodiments, the method further can include administering an anti-dyskensia agent and/or anti-psychotic agent. Suitable anti-dyskenia agents include baclofen (such as Lioresal™), botulinum toxin (such as BotoX™), clonazepam (such as Klonopin™), and diazepam (such as Valium™). Exemplary antipsychotic agents are described herein.

Embodiments disclosed herein relate to a method of alleviating or treating schizophrenia that can include administering a therapeutically effective amount of at least one isolated form of a compound described herein or a pharmaceutical composition described herein to a subject suffering from schizophrenia.

An embodiment disclosed herein relates to a method of alleviating or treating migraine that can include administering a therapeutically effective amount of at least one isolated form of a compound described herein or a pharmaceutical composition described herein to a subject who suffers from a migraine.

Some embodiments disclosed herein relate to a method of alleviating or treating psychosis that can include administering a therapeutically effective amount of at least one isolated form of a compound described herein or a pharmaceutical composition described herein to a subject suffering from psychosis. As stated previously, the psychosis can be caused or results from various different origins. In an embodiment, the psychosis can be selected from drug-induced psychosis, treatment-induced psychosis and psychosis associated with a disease. Examples of diseases which are associated with psychosis include dementia, post traumatic stress disorder, Alzheimer's disease, Parkinson's disease and schizophrenia.

Embodiments disclosed herein relate to a method of alleviating or treating a condition amenable for treatment with an antipsychotic that can include administering a first amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein), and a second amount of an anti-psychotic compound to a subject, wherein the second amount of the anti-psychotic compound is less than the amount of the anti-psychotic compound needed to produce a comparable efficacious effect when the anti-psychotic compound is administered alone. In some embodiments, the first amount and the second amount can be co-administered. In an embodiment, the co-administration can result in decreased severity or slower onset of a side effect associated with the antipsychotic agent as compared to the administration of the amount of the anti-psychotic agent alone, Exemplary antipsychotic compounds are described herein.

Some embodiments disclosed herein relate to a method of alleviating or treating a pituitary tumor that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject with a pituitary tumor. In an embodiment, the tumor can be a prolactinoma. An embodiment disclosed herein relates to a method of inhibiting the formation of a pituitary tumor that can include administering a therapeutically effective amount of at least one isolated form of a compound described herein or a pharmaceutical composition described herein to a subject at risk for forming a pituitary tumor. Embodiments disclosed herein relate to a method of reducing the level of prolactin in a subject that can include administering a therapeutically effective amount of at least one isolated form of a compound described herein or a pharmaceutical composition described herein to a subject with elevated levels of prolactin.

An embodiment disclosed herein relates to a method of reducing or inhibiting weight gain that can include administering a therapeutically effective amount of at least one isolated form of a compound described herein (e.g., an isolated form of a compound of Formulae (I), (II), (III), (IV) and/or AV) as described herein) or a pharmaceutical composition described herein (e.g., a pharmaceutical composition that includes an effective amount of an isolated form of a compound of Formulae (I), (II), (III), (IV) and/or (V) as described herein) to a subject at risk of gaining weight. In an embodiment, the subject can be at risk to gain weight due to being administered a drug (e.g., an antipsychotic) that causes weight gain.

Embodiments disclosed herein relate to a method of alleviating or treating a sleep disorder that can include administering a therapeutically effective amount of at least one isolated form of a compound described herein (e.g., an isolated form of a compound of Formulae (I), (II), (III), (IV) and/or (V) as described herein) or a pharmaceutical composition described herein (e.g., a pharmaceutical composition that includes an effective amount of an isolated form of a compound of Formulae (I), (II), (III), (IV) and/or (V) as described herein) to a subject suffering from a sleep disorder. In some embodiments, the sleep disorder can be insomnia such as sleep maintenance insomnia. Some embodiments disclosed herein relate to a method of increasing slow-wave sleep that can include administering a therapeutically effective amount of at least one isolated form of a compound described herein or a pharmaceutical composition described herein to a subject. An embodiments disclosed herein relates to a method of alleviating or treating insomnia that can include administering a sleep-inducing agent adapted to induce onset of sleep in a subject; and administering to the subject a therapeutically effective amount of at least one isolated form of a compound described herein or a pharmaceutical composition described herein to maintain the sleep induced by the sleep-inducing agent. Embodiments disclosed herein relate to a method of alleviating or treating sleep maintenance insomnia that can include administering a therapeutically effective amount of at least one isolated form of a compound described herein or a pharmaceutical composition described herein to a subject suffering from sleep maintenance insomnia at a frequency of every other day or greater.

An embodiment disclosed herein relates to a method for identifying a compound which binds to a serotonin receptor that can include labeling an isolated form of a compound described herein; with a detectable label; contacting the serotonin receptor with the labeled compound; and determining whether the labeled compound binds to the serotonin receptor. In an embodiment, the detectable label can be a radiolabel such as [³H], [¹⁸F], [¹¹C] and [¹²⁵I].

In some embodiments, the compounds disclosed herein are potent inverse agonist and/or antagonists of a serotonin receptor. In an embodiment, the serotonin receptor is a 5-HT2A receptor. In an embodiment, the serotonin receptor is a 5-HT2C receptor. In an embodiment, the compounds described herein have found to not interact strongly with other serotonin receptors (5-HT 1A, 1B, 1D, 1E, 1F, 2B, 4A, 6, and 7) at concentrations where the signaling of the 5-HT2A and/or 5-HT2C receptors is strongly or completely inhibited. In one embodiment, the compound is also selective with respect to other monoamine-binding receptors, such as the dopaminergic, histaminergic, adrenergic and muscarinic receptors. Compounds that are selective for 5-HT2A and/or 5-HT2C receptors may have a beneficial effect in the treatment of psychosis, schizophrenia or similar neuropsychiatric disorders, while avoiding adverse effects associated with drugs hitherto suggested for this purpose.

### EXAMPLES

Chemistry. ¹H NMR spectra were recorded at 400 MHz on a Varian Mercury-VX400 MHz spectrometer and chemical shifts are given in δ-values [ppm] referenced to the residual solvent peak chloroform (CDCl₃) at 7.26 and methanol (CD₃OD) at 3.31 ppm. Coupling constants, J, are reported in Hertz. Unless otherwise stated, the NMR spectra of the compounds are described for their free amine form. Column chromatography was carried out using silica gel 60 (particle size 0.030-0.070 mm) from Merck. Materials and solvents were of the highest grade available from commercial sources and used without further purification. Reversed phase C₁₈ solid phase extraction cartridges (SPE) were DSC-18 2 g/12 mL columns from Discovery™ Solid Phase Extraction Products, Supelco. Preparative HPLC was run on a Waters/Micromass HPLC/MS using a diode array detector (190-450 nm) UV detector and Micromass ZMD-mass-spectrometer with electrospray ionization. A YMC J'sphere ODS H80 19×100 mm column was used. The mobile phase was 0.15% TFA in water/acetonitrile with a gradient starting at 30% acetonitrile, going to 100% acetonitrile over 13 min. The flow rate was 17 mL/min.

HPLC/LCMS Method. Samples were run on a Waters/Micromass HPLC/MS using a diode array detector (190-450 nm) UV detector and Micromass ZMD-mass-spectrometer with electrospray ionization. A Phenomenex Luna C₁₈(2) 3 μm, 75×4.6 mm column was used. The mobile phase was 10 mM ammonium acetate in water/acetonitrile with a gradient starting at 30% acetonitrile, going to 95% acetonitrile over 12 min. The flow rate was 1.0 mL/min.

Preparation of Hydrochloride Salts. the Tertiary Amine Products were dissolved in dichloromethane, treated with an excess of 1M HCl in diethyl ether and precipitated from n-heptane. The solvents were removed in vacuo and after drying, the hydrochloride salts were obtained as colorless solids in quantitative yield.

### N-(4-Fluorobenzyl)-N-(1-methylpiperidin-4-yl)-N′-(4-hydroxybenzyl)carbamide Hydrochloride

N-((4-Fluorophenyl)methyl)-4-amino-1-methylpiperidine was prepared from 1-methylpiperidine-4-one (1.15 mL, 10 mmol), which was dissolved in methanol (30 mL). 4-Fluorobenzylamine (1.25 mL, 10 mmol) was added and the pH was adjusted to 5 with acetic acid. NaBH₃CN (1.25 g, 20 mmol) was added and the reaction mixture was stirred for 3 h, after which it was concentrated. 2M aqueous NaOH (30 mL) was added and the mixture extracted with dichloromethane (2×50 mL). The combined organic phases were dried over Na₂SO₄, filtered and evaporated, and this crude product was purified by Kugelrohr distillation to give the desired product (1.1 g, 50%) as a clear oil.

N-(4-Fluorobenzyl)-4-amino-1-methylpiperidine (4.00 g, 18.0 mmol) was dissolved in dichloromethane (150 mL). 4-Methoxybenzyl isocyanate (3.26 g, 20.0 mmol) in dichloromethane (50 mL) was added dropwise and the mixture was stirred for 3 h at room temperature. The crude mixture was concentrated and purified by flash chromatography (0-10% methanol in dichloromethane) to give N-((4-fluorophenyl)methyl)-N-(1-methylpiperidin-4-yl)-N′-((4-methoxyphenyl)methyl)carbamide (4.91 g, 71%). This carbamide (4.91 g, 13.0 mmol) was dissolved in dry dichloromethane (50 mL). The solution was cooled to 0° C. and boron tribromide (1M in dichloromethane, 39.0 mL, 39.0 mmol) was added dropwise, and the mixture stirred for 20 h at room temperature. Water (50 mL) and n-butanol (10 mL) were added and the phases separated. The aqueous phase was extracted a second time with a mixture of dichloromethane (50 mL) and n-butanol (10 mL). The combined organic phases were evaporated and the resulting solid was purified by flash chromatography (0-20% methanol in dichloromethane) to give a semi-pure solid (3.17 g, 67%). An analytical amount (25 mg) of this material was purified by preparative HPLC to give a colorless oil (10 mg). LC-MS showed [M+H]⁺=372 (characteristic fragment: 223). ¹H-NMR (CD₃OD, 400 MHz, Free base): δ 7.25-6.62 (m, 8H), 4.46 (s, 2 μl), 4.22 (s, 2H), 4.15-4.06 (m, 1H), 2.89-2.82 (m, 2H), 2.23 (s, 3H), 2.14-2.05 (m, 2H), 1.74-1.61 (m, 4H).

The collected compound was converted into its hydrochloride salt, which was obtained as a colorless solid.

### N-(4-Fluorobenzyl)-N-(piperidin-4-yl)-N′-(4-isobutoxybenzyl)carbamide Hydrochloride

4-Piperidone hydrochloride monohydrate (4.0 g, 26.0 mmol) was dissolved in dichloromethane (130 mL). After addition of triethylamine (8.66 g, 85.8 mmol) the mixture was stirred for 10 min and then cooled to 0° C. Trifluoroacetic anhydride (12.0 g, 57.2 mmol) was added dropwise under stirring. After 2 hours at room temperature, the reaction was stopped by addition of water (100 mL). The aqueous phase was extracted with dichloromethane (2×100 mL). The combined organic phases were dried over Na₂SO₄, filtered and concentrated to give 1-trifluoroacetyl-4-piperidone (5.07 g, 100%). 4-Fluorobenzylamine (3.14 g, 25.9 mmol) and 1-trifluoroacetyl-4-piperidone (5.07 g, 25.9 mmol) were added to a solution of methanol adjusted to pH 5 with acetic acid (150 mL). The reaction mixture was stirred for 5 min and NaBH₃CN (2.46 g, 38.9 mmol) was added slowly under stirring. After 20 hours at room temperature the reaction was concentrated. 2M aqueous NaOH (100 mL) was added and extracted with dichloromethane (2×100 mL). The combined organic phases were dried over Na₂SO₄, filtered and concentrated to give N-(4-fluorobenzyl)-4-amino-1-trifluoroacetylpiperidine (2.91 g, 37%).

4-Isobutoxybenzyl isocyanate was prepared from 4-isobutoxyphenylacetic acid (7.6 g, 36.5 mmol) (prepared according to classical literature procedures from methyl 4-hydroxyphenylacetate by a Williamson ether synthesis with isobutylbromide, followed by saponification of the ester. For an alternative route see: Profft; Drux; J. Prakt. Chem. 1956, 4(3), 274-275), which is hereby incorporated by reference in its entirety, and which was dissolved in THF (50 mL). Proton Sponge™ (8.2 g, 38 mmol) was added, and the mixture was stirred for 15 min. Diphenylphosphoryl azide (10.6 g, 38 mmol) was added dropwise and the mixture was heated to reflux for 4 h. The mixture was cooled to room temperature and placed in the freezer at −18° C. for 20 h. The resulting white precipitate was vigorously stirred with diethyl ether (250 mL) for 15 min and filtered. The filtrate was evaporated to give the desired product, which was used without further purification.

N-(4-fluorobenzyl)-4-amino-1-trifluoroacetylpiperidine (2.91 g, 9.6 mmol) was dissolved in dichloromethane (50 mL) and a solution of 4-isobutoxybenzyl isocyanate (1.97 g, 9.6 mmol) in dichloromethane (50 mL) was added. The reaction mixture was stirred for 20 h and concentrated. The crude product was purified by flash chromatography (0-5% methanol in dichloromethane) to give N-(4-fluorobenzyl)-N-(1-trifluoroacetylpiperidin-4-yl)-N′-(4-isobutoxybenzyl)carbamide (3.90 g, 91%).

This carbamide (3.90 g, 8.7 mmol) was dissolved in methanol (12 mL) and added to a 2M solution of potassium carbonate in methanol (100 mL) under stirring. After 4 hours the methanol was evaporated, and the aqueous phase was extracted with dichloromethane (2×100 mL). The combined organic phases were dried over Na₂SO₄, filtered and concentrated to give a semi-pure solid (2.95 g, 85%). An analytical amount (200 mg) of this crude product was purified by flash chromatography (10% methanol in dichloromethane with 1% triethylamine) to give a colorless solid (100 mg). LC-MS showed [M+H]⁺=414 (characteristic fragment: 209). ¹H-NMR (CDCl₃, 400 MHz, Free base): δ 7.21-6.75 (m, 8H), 4.47-4.42 (m, 1H), 4.39 (t, J=5.0 Hz, 1H), 4.35 (s, 2H), 4.27 (d, J=5.0 Hz, 2H), 3.68 (d, J=6.0 Hz, 2H), 3.13-3.06 (m, 2H), 2.74-2.66 (m, 2H), 2.11-1.99 (m, 1H), 1.78-1.71 (m, 3H), 1.58-1.46 (m, 2H), 1.00 (d, J=6.0 Hz, 6H).

The collected compound was converted into its hydrochloride salt, which was obtained as a colorless solid.

### N-(4-Fluorobenzyl)-N-(1-methylpiperidin-4-yl)-N′-[4-(2-hydroxy)isobutoxybenzyl]carbamide Hydrochloride

N-(4-Fluorobenzyl)-N-(1-methylpiperidin-4-yl)-N′-(4-hydroxybenzyl) carbamide (375 mg, 1.0 mmol) was dissolved in DMF (15 mL). KOH (281 mg, 5.0 mmol) was added and the mixture was stirred 30 min at room temperature. Isobutylene oxide (216 mg, 3.0 mmol) was added and the mixture was warmed to 40° C. for 20 h. Isobutylene oxide (216 mg, 3.0 mmol) was added and the mixture was stirred at 40° C. for another 20 h. Water (50 mL) was added and the mixture was extracted with dichloromethane (2×60 mL). The combined organic phases were dried over Na₂SO₄, filtered and evaporated. The crude product was purified by flash chromatography (5% methanol in dichloromethane) and subsequently by passage over a C₁₈-SPE cartridge, eluting with 30% acetonitrile/water and 3.5 mM ammonium acetate buffer. The acetonitrile was evaporated and the water phase was made alkaline with aqueous ammonia. The product was extracted into dichloromethane (2×100 mL), and the combined organic phases were dried over Na₂SO₄, filtered and evaporated to give a colorless oil (122 mg, 28%). LC-MS showed [M+H]⁺=444 (characteristic fragment: 223). ¹H-NMR (CDCl₃, 400 MHz, Free base): δ 7.21-6.77 (m, 8H), 4.49-4.43 (t, J=5.5 Hz, 1H), 4.37-4.26 (m, 5H), 3.75 (s, 2H), 2.89-2.82 (m, 2H), 2.25 (s, 3H), 2.10-2.01 (m, 2H), 1.76-1.58 (m, 4H), 1.33 (s, 6H).

The collected compound was converted into its hydrochloride salt, which was obtained as a colorless solid.

### N-(4-Fluorobenzyl)-N-(1-methyl-piperidin-4-yl)-N′-(4-(R)-[(3-hydroxy)-isobutoxy]benzyl)carbamide

N-(4-Fluorobenzyl)-N-(1-methylpiperidin-4-yl)-N′-(4-hydroxybenzyl)carbamide (75 mg, 0.20 mmol) was dissolved in DMF (3 mL). Potassium hydroxide (56 mg, 1.00 mmol) was added and the mixture was stirred 15 minutes at room temperature. (R)-(−)-3-Bromo-2-methyl-1-propanol (93 mg, 0.60 mmol) was added. The mixture was heated to 60° C. for 5 hours. The reaction mixture was cooled to room temperature, added to dichloromethane (50 mL), and washed with 1M potassium hydroxide (50 mL). The organic phase was dried over Na₂SO₄, filtered and evaporated. The resulting oil was purified by preparative HPLC to give N-(4-Fluorobenzyl)-N-(1-methyl-piperidin-4-yl)-N′-(4-(R)-[(3-hydroxy)-isobutoxy]benzyl)carbamide as a colorless oil (5 mg, 6%). LC-MS showed [M+H]⁺=444 (characteristic fragment: 223). ¹H-NMR (CDCl₃, 400 MHz, free base): δ 7.20-6.78 (m, 8H), 4.47 (t, J=5 Hz, 1H), 4.35-4.29 (m, 3H), 4.27 (d, J=5.0 Hz, 2H), 3.92-3.89 (m, 2H), 3.68 (d, J=6.0 Hz, 2H), 2.89-2.82 (m, 2H), 2.25 (s, 3H), 2.22-2.13 (m, 1H), 2.10-2.02 (m, 2H), 1.83-1.76 (bs, 1H), 1.75-1.59 (m, 4H) 1.02 (d, J=6.0 Hz, 3H).

The collected compound was converted into its hydrochloride salt, which was obtained as a colorless solid.

### N-(4-Fluorobenzyl)-N-(1-methyl-1-oxopiperidin-4-yl)-N′-(4-isobutoxybenzyl)carbamide

N-(4-Fluorobenzyl)-N-(1-methylpiperidin-4-yl)-N′-(4-isobutoxybenzyl)carbamide (100 mg, 0.234 mmol) was dissolved in dichloromethane (10 mL). The solution was cooled to 0° C. and meta-chloroperbenzoic acid (57-86%, 106 mg, 0.351 mmol) was added. The reaction mixture was stirred for 20 h at room temperature, after which it was washed with saturated aqueous NaHCO₃ (10 mL). The organic phase was dried over Na₂SO₄, filtered and evaporated. The resulting oil was purified by preparative HPLC to give N-(4-fluorobenzyl)-N-(1-methyl-1-oxopiperidin-4-yl)-N′-(4-isobutoxybenzyl)carbamide as a colorless oil (10 mg, 10%). LC-MS showed [M+H]⁺=444 (characteristic fragment: 239). ¹H-NMR (CDCl₃, 400 MHz): δ 7.20-6.76 (m, 8H), 4.63-4.53 (m, 2H), 4.43 (s, 2H), 4.24 (d, J=5.0 Hz, 2H), 3.66 (d, J=7.0 Hz, 2H), 3.31-3.24 (m, 4H), 3.19 (s, 3H), 2.62-2.51 (m, 2H), 2.10-1.99 (m, 1H), 1.69-1.62 (m, 2H), 1.00 (d, J=7.0 Hz, 6H).

### In Vitro Determination of Receptor Activity

Receptor Selection and Amplification (R-SAT®) Assays. The functional receptor assay, Receptor Selection and Amplification Technology (R-SAT®), was used (with minor modifications from the procedure described previously (Brann, M. R. U.S. Pat. No. 5,707,798, 1998; Chem. Abstr. 1998, 128, 111548) to screen compounds for efficacy at the 5-HT_{2A} receptor. Briefly, NIH3T3 cells were grown in 96 well tissue culture plates to 70-80% confluence. Cells were transfected for 12-16 h with plasmid DNAs using superfect (Qiagen Inc.) as per manufacturer's protocols. R-SAT's were generally performed with 50 ng/well of receptor and 20 ng/well of β-galactosidase plasmid DNA. All receptor and G-protein constructs used were in the pSI mammalian expression vector (Promega Inc) as described previously. The 5-HT_{2A} or 5-HT_{2C} receptor gene was amplified by nested PCR from brain cDNA using the oligodeoxynucleotides based on the published sequence (Saltzman et. al, Biochem. Biophys. Res. Comm. 1991, 181, 1469). For large-scale transfections, cells were transfected for 12-16 h, then trypsinized and frozen in DMSO. Frozen cells were later thawed, plated at 10,000-40,000 cells per well of a 96 well plate that contained drug. With both methods, cells were then grown in a humidified atmosphere with 5% ambient CO₂ for five days. Media was then removed from the plates and marker gene activity was measured by the addition of the β-galactosidase substrate o-nitrophenyl β-D-galactopyranoside (ONPG, in PBS with 5% NP-40). The resulting colorimetric reaction was measured in a spectrophotometric plate reader (Titertek Inc.) at 420 nM. All data were analyzed using the computer program XLFit (IDBSm). Efficacy is the percent maximal repression compared to repression by a control compound (ritanserin in the case of 5-HT_{2A}). pIC₅₀ is the negative of the log(IC₅₀), where IC₅₀ is the calculated concentration in Molar that produces 50% maximal repression. Various metabolites of N-(1-methylpiperidin-4-yl)-N-(4-fluorophenylmethyl)-N′-(4-(2-methylpropyloxy)phenylmethyl) carbamide, including the compounds of Formulae (I), (II), (III), (IV) and (V) as well as other metabolites, were assayed as described herein. The assayed metabolites demonstrated varying activity levels with some of the metabolites exhibiting levels too low for use as pharmaceuticals agents. Compounds of Formulae (I), (II), (III), (IV) and (V), however, demonstrated high inverse agonist and antagonist activity as shown in the table below. This data indicates compounds of Formulae (I), (II), (III), (IV) and (V) could be useful as pharmaceutical agents.

| | |
|---|---|
| Inverse Agonist | Antagonist |
| pIC₅₀ | pKi |

| Compound | 5HT2A | 5HT2C | 5HT2A | 5HT2C |
|---|---|---|---|---|
| Formula (I) | 7.5 | — | 7.9 | 6.5 |
| Formula (II) | 8.6 | — | 8.9 | 7 |
| Formula (III) | 8.7 | 6.1 | 9 | 6.8 |
| Formula (IV) | 8.5 | 6.7 | — | 6.8 |
| Formula (V) | 7.5 | 5.7 | 7.5 | 6.4 |

It will be understood by those of skill in the art that numerous and various modifications can be made without departing from the spirit of the present disclosure. Therefore, it should be clearly understood that the forms disclosed herein are illustrative only and are not intended to limit the scope of the present disclosure.

What is claimed is:

1. A isolated form of a compound selected from the group consisting of Formulae (I), (II), (III), (IV) and (V), wherein the compounds of Formulae (I), (II), (III), (IV) and (V) have the following structures: 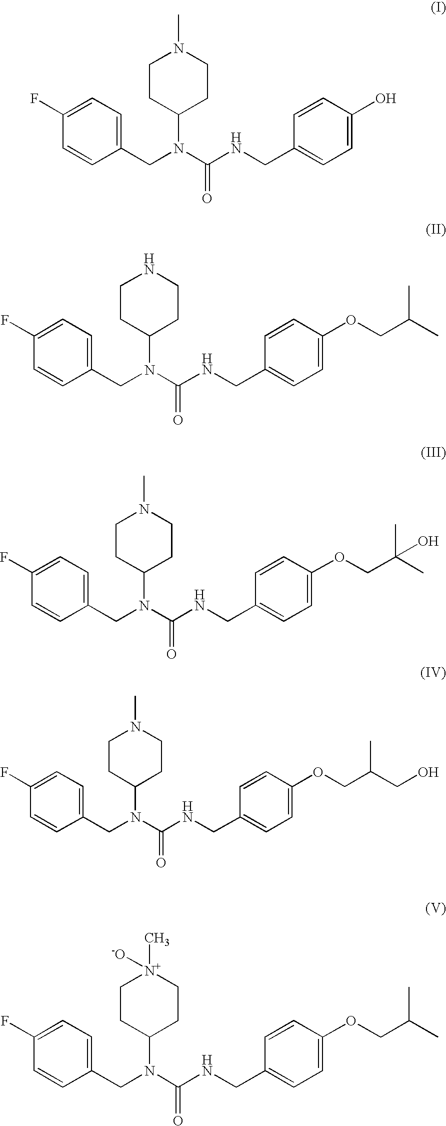
or a pharmaceutically acceptable salt, prodrug, hydrate, solvate, polymorph, or ester thereof.

2. A pharmaceutical composition comprising at least one isolated form of the compound of claim 1, and a pharmaceutically acceptable carrier, diluent, or excipient.

3. The pharmaceutical composition of claim 2, wherein the pharmaceutical composition is suitable for oral administration.

4. The pharmaceutical composition of claim 2, further comprising an additional therapeutic agent.

5. The pharmaceutical composition of claim 4, wherein the additional therapeutic agent is selected from the group consisting of a dopaminergic agent, anti-dyskensia agent, anti-dystonia agent, anti-myoclonus agent, anti-tremor agent, anti-psychotic agent, antidepressant, anti-dementia agent and sleep-inducing agent.

6. The pharmaceutical composition of claim 5,
wherein the dopaminergic agent is selected from the group consisting of levodopa, bromocriptine, pergolide, ephenedrine sulfate, pemoline, mazindol, d,1-α-methylphenethylamine, methylphenydate, pramipexole, modafinil, and ropinirole;
wherein the anti-dyskensia agent, anti-dystonia, anti-myoclonus, or anti-tremor agent is selected from the group consisting of baclofen, botulinum toxin, clonazepam, and diazepam;
wherein the anti-psychotic agent is selected from the group consisting of chlorpromazine, haloperidol, molindone, thioridazine, a phenothiazine, a butyrophenome, a phenylbutylpiperadine, thioxanthine, a substituted benzamide, sertindole, amisulpride, risperidone, clozapine, olanzapine, ziprasidone, a debenzapine, a benzisoxidil, a salt of lithium, Aripiprazole, Etrafon®, Droperidol, Thioridazine, Thiothixene, Promethazine, Metoclopramide, Chlorprothixene, Triavil®, Molindone, Sertindole, Amisulpride, Melperone, Paliperidone, Tetrabenazine and their active metabolites;
wherein the phenothiazine is selected from the group consisting of chlorpromazine, mesoridazine, prochlorperazine, thioridazine, Fluphenazine, Perpehnazine, and Trifluoperazine;
wherein the phenylbutylpiperadine is pimozide;
wherein the debenzapine is selected from the group consisting of clozapine, loxapine, olanzapine, and quetiapine;
wherein the benzisoxidil is ziprasidone;
wherein the salt of lithium is lithium carbonate;
wherein the antidepressant is selected from the group consisting of citalopram, escitalopram oxalate, fluoxetine, fluvoxamine maleate, paroxetine, sertraline, and dapoxetine;
wherein the anti-dementia agent is a cholinesterase inhibitor; and
wherein the sleep-inducing agent is selected from the group consisting of zolpidem, eszopiclone, a benzodiazepine, a melatonin agonist, and an antihistamine.

7. The pharmaceutical composition of claim 6, wherein the cholinesterase inhibitor is selected from the group consisting of are donepezil, galantamine, rivastigmine, tacrine, metrifonate, physostigmine, neostigmine, pyridostigmine, ambenonium, demarcarium, aldicarb, bendiocarb, bufencarb, carbaryl, carbendazim, carbetamide, carbofuran, chlorbufam, chloropropham, ethiofencarb, formetanate, methiocarb, methomyl, oxamyl, phenmedipham, pinmicarb, pirimicarb, propamocarb, propham, propoxur, edrophonium, phenothiazines, echothiophate, diisopropyl fluorophosphate, dimebon, Huperzine A, T-82 ((2-[2-(1-benzylpiperidin-4-yl)ethyl]-2,3-dihydro-9-methoxy-1H-pyrrolo[3,4-b]quinolin-1-one hemifumarate)), TAK-147 (zanapezil), phenserine, quilostigmine, ganstigmine, butyrophenones, imipramines, tropates, phencyclidines, curariforms, ethephon, ethopropazine, iso-OMPA, tetrahydrofurobenzofuran cymserine, N¹phenethyl-norcymserine, N⁸-benzylnorcymserine, N¹, N⁸-bisnorcymserine, N¹-N⁸-bisbenzylnorphysostigmine, N¹,N⁸-bisbenzylnorphenserine and N¹, N⁸-bisbenzylnorcymserine; wherein the benzodiazepine is selected from the group consisting of temazepam, diazepam, lorazepam, nitrazepam, and midazolam; wherein the melatonin agonist is ramelteon; and wherein the antihistamine is diphenhydramine.

8. A method of inhibiting the activity of a serotonin receptor comprising contacting the monoamine receptor or a system containing a monoamine receptor with at least one isolated form of a compound of claim 1 or a pharmaceutical composition comprising at least one isolated form of said compound of claim 1.

9. The method of claim 8, wherein the serotonin receptor is a subclass selected from the group consisting of in the 5-HT2A subclass and 5-HT2C subclass.

10. The method of claim 8, wherein the serotonin receptor is mutated or modified.

11. A method of inhibiting an activation of a serotonin receptor comprising contacting the monoamine receptor of a system containing a monoamine receptor with at least one isolated form of a compound of claim 1 or a pharmaceutical composition comprising at least one isolated form of said compound of claim 1.

12. The method of claim 11, wherein the serotonin receptor is a subclass selected from the group consisting of in the 5-HT2A subclass and 5-HT2C subclass.

13. The method of claim 11, wherein the serotonin receptor is mutated or modified.

14. A method of alleviating or treating one or more disease condition associated with a serotonin receptor comprising administering a therapeutically effective amount of at least one isolated form of a compound of claim 1 or a pharmaceutical composition comprising at least one isolated form of said compound of claim 1.

15. The method of claim 14, wherein the disease condition is a neuropsychiatric disorder.

16. The method of claim 15, wherein the neuropsychiatric disorder is selected from the group consisting of schizophrenia, schizoaffective disorder, mania, depression, a cognitive disorder, aggressiveness, panic attacks, obsessive compulsive disorder, borderline personality disorder, borderline disorder, multiplex developmental disorder (MDD), a behavioral disorder, psychosis, suicidal tendency, bipolar disorder, sleep disorder, addiction, attention deficit hyperactivity disorder (ADHD), post traumatic stress disorder (PTSD), Tourette's syndrome, anxiety, autism, Down's syndrome, a learning disorder, a psychosomatic disorder, alcohol withdrawal, epilepsy, pain, a disorder associated with hypoglutamatergia, and serotonin syndrome.

17. The method of claim 16, wherein the psychosis is selected from drug-induced psychosis, treatment-induced psychosis and psychosis associated with a disease.

18. The method of claim 17, wherein the disease is dementia, post traumatic stress disorder, Alzheimer's disease, Parkinson's disease and schizophrenia.

19. The method of claim 14, wherein the disease condition is a neurodegenerative disorder.

20. The method of claim 19, wherein the neurodegenerative disorder is selected from Alzheimer's disease, Parkinson's disease, Huntington's chorea, sphinocerebellar atrophy, frontotemporal dementia, supranuclear palsy and Lewy body dementia.

21. The method of claim 14, wherein the disease condition is selected from the group consisting of chemotherapy-induced emesis, frailty, on/off phenomena, non-insulin-dependent diabetes mellitus, metabolic syndrome, an autoimmune disorder, sepsis, increased intraocular pressure, glaucoma, a retinal disease, Charles Bonnet syndrome, substance abuse, sleep apnea, pancreatis, anorexia, bulimia, a disorder associated with alcoholism, a cerebral vascular accident, amyotrophic lateral sclerosis, AIDS related dementia, traumatic brain, traumatic spinal injury, tinnitus, a menopausal symptom, sexual dysfunction, low male fertility, low sperm motility, hair loss, hair thinning, incontinence, hemorrhoids, migraine, hypertension, thrombosis, abnormal hormonal activity, a hormonal disorder, a pituitary tumor, a side effect associated with a pituitary tumor, vasospasm, ischemia, cardiac arrhythmia, cardiac insufficiency, asthma, emphysema, and an appetite disorder.

22. The method of claim 14, wherein the disease condition is associated with dysfunction of the serotonin receptor, activation of the serotonin receptor or increased activity of the serotonin receptor.

23. The method of claim 14, wherein the serotonin receptor is a subclass selected from the group consisting of in the 5-HT2A subclass and 5-HT2C subclass.

24. The method of claim 14, wherein the serotonin receptor is mutated or modified.

25. The method of claim 14, comprising administering an additional therapeutic agent.

26. The method of claim 25, wherein the additional therapeutic agent is a dopaminergic agent, anti-dyskensia agent, anti-dystonia agent, anti-myoclonus agent, anti-tremor agent, anti-psychotic agent, antidepressant, anti-dementia agent, or sleep-inducing agent.

27. The method of claim 26, wherein the dopaminergic agent is selected from the group consisting of levodopa, bromocriptine, pergolide, ephenedrine sulfate, pemoline, mazindol, d,1-α-methylphenethylamine, methylphenydate, pramipexole, modafinil, and ropinirole; wherein the anti-dyskensia agent, anti-dystonia agent, anti-myoclonus agent, or anti-tremor agent is selected from the group consisting of baclofen, botulinum toxin, clonazepam, and diazepam; wherein the anti-psychotic agent selected from the group consisting of chlorpromazine, haloperidol, molindone, thioridazine, a phenothiazine, a butyrophenome, a phenylbutylpiperadine, thioxanthine, sulpiride, sertindole, amisulpride, risperidone, clozapine, olanzapine, ziprasidone, a debenzapine, a benzisoxidil, a salt of lithium, Aripiprazole, Etrafon®, Droperidol, Thioridazine, Thiothixene, Promethazine, Metoclopramide, Chlorprothixene, Triavil®, Molindone, Sertindole, Amisulpride, Melperone, Paliperidone, Tetrabenazine and their active metabolites; wherein the antidepressant is selected from the group consisting of citalopram, escitalopram oxalate, fluoxetine, fluvoxamine maleate, paroxetine, sertraline, and dapoxetine; wherein the anti-dementia agent is a cholinesterase inhibitor; and wherein the sleep-inducing agent is selected from the group consisting of zolpidem, eszopiclone, a benzodiazepine, a melatonin agonist, and an antihistamine.

28. The method of claim 27, wherein the phenothiazine is selected from the group consisting of chlorpromazine, mesoridazine, prochlorperazine, thioridazine, Fluphenazine, Perpehnazine, and Trifluoperazine; wherein the phenylbutylpiperadine is pimozide; wherein the debenzapine is selected from the group consisting of clozapine, loxapine, olanzapine, and quetiapine; wherein the benzisoxidil is ziprasidone; wherein the salt of lithium is lithium carbonate; wherein the cholinesterase inhibitor is selected from the group consisting of are donepezil, galantamine, rivastigmine, tacrine, metrifonate, physostigmine, neostigmine, pyridostigmine, ambenonium, demarcarium, aldicarb, bendiocarb, bufencarb, carbaryl, carbendazim, carbetamide, carbofuran, chlorbufam, chloropropham, ethiofencarb, formetanate, methiocarb, methomyl, oxamyl, phenmedipham, pinmicarb, pirimicarb, propamocarb, propham, propoxur, edrophonium, phenothiazines, echothiophate, diisopropyl fluorophosphate, dimebon, Huperzine A, T-82 ((2-[2-(1-benzylpiperidin-4-yl)ethyl]-2,3-dihydro-9-methoxy-1H-pyrrolo[3,4-b]quinolin-1-one hemifumarate)), TAK-147 (zanapezil), phenserine, quilostigmine, ganstigmine, butyrophenones, imipramines, tropates, phencyclidines, curariforms, ethephon, ethopropazine, iso-OMPA, tetrahydrofurobenzofuran cymserine, N¹phenethyl-norcymserine, N⁸-benzylnorcymserine, N¹, N⁸-bisnorcymserine, N¹-N⁸-bisbenzylnorphysostigmine, N¹, N⁸-bisbenzylnorphenserine and N¹, N⁸-bisbenzylnorcymserine; wherein the benzodiazepine is selected from the group consisting of temazepam, diazepam, lorazepam, nitrazepam, and midazolam; wherein the melatonin agonist is ramelteon; and wherein the antihistamine is diphenhydramine.

29. A method of alleviating or treating a condition induced by the administration of an anti-psychotic compound comprising administering a therapeutically effective amount of at least one isolated form of a compound of claim 1 or a pharmaceutical composition comprising at least one isolated form of said compound of claim 1 to a subject being administered the anti-psychotic compound.

30. The method of claim 29, wherein the condition is a side effect selected from the group consisting of an extrapyramidal side effect, a histaminic side effect, an alpha adrenergic side effect, and an anticholinergic side effect.

31. The method of claim 29, wherein the condition is selected from the group consisting of stroke, tremors, sedation, gastrointestinal problems, neurological problems, increased risk of death, a cerebrovascular event, a movement disorder, dystonia, akathisia, a parkinsoniam movement disorder, dyskinesia, tardive dyskinesia, a cognitive disorder, prolactinemia, catalepsy, psychosis, neuroleptic malignant syndrome, a heart problem, a pulmonary problem, diabetes, liver failure, suicidality, sedation, orthostatic hypotension, choking, dizziness, tachycardia, blood abnormalities, an abnormal triglyceride level, an increased cholesterol level, dyslipidemia, hyperglycemia, syncope, a seizure, dysphagia, priapism, thrombotic thrombocytopenic purpura, disruption of body temperature regulation, insomnia, agitation, anxiety, somnolence, aggressive reaction, headache, constipation, nausea, dyspepsia, vomiting, abdominal pain, saliva increase, toothache, rhinitis, coughing, sinusitis, pharyngitis, dyspnea, back pain, chest pain, fever, rash, dry skin, seborrhea, increased upper respiratory infection, abnormal vision, arthralgia, hypoaesthesia, manic reaction, concentration impairment, dry mouth, pain, fatigue, acne, pruritus, myalgia, skeletal pain, hypertension, diarrhea, confusion, asthenia, urinary incontinence, sleepiness, increased duration of sleep, accommodation disturbance, palpitations, erectile dysfunction, ejaculatory dysfunction, orgastic dysfunction, lassitude, increased pigmentation, increased appetite, automatism, increased dream activity, diminished sexual desire, nervousness, depression, apathy, catatonic reaction, euphoria, increased libido, amnesia, emotional liability, a nightmare, delirium, yawning, dysarthria, vertigo, stupor, paraesthesia, aphasia, hypoesthesia, tongue paralysis, a leg cramp, torticollis, hypotonia, coma, migrain, hyperreflexia, choreoathetosis, anorexia, flatulence, stomatitis, melena, hemorrhoids, gastritis, fecal incontinence, erutation, gastroeophageal reflux, gastroenteritis, esophagitis, tongue discoloration, choleithiasis, tongue edema, diverticulitis, gingivitis, discolored feces, gastrointestinal hemorrhage, hematemesis, edema, rigors, malaise, pallor, enlarged abdomen, ascites, sarcoidosis, flushing, hyperventilation, bronchospasm, pneumonia, tridor, asthma, increased sputum, aspiration, photosensitivity, increased sweating, acne, descreased sweating, alopecia, hyperkeratosis, skin exfoliation, bullous eruption, skin ulceration, aggravated psoriasis, furunculosis, verruca, dermatitis lichenoid, hypertrichosis, genital pruritus, urticaria, ventricular tachycardia, angina pectoris, premature atrial contractions, T wave inversion, a ventricular extrasystole, ST depression, AV block, myocarditis, abnormal accommodation, xerophthalmia, diplopia, eye pain, blepharitis, photopsia, photophobia, abnormal lacrimation, hyponatremia, creatine phosphokinase increase, thirst, weight decrease, decreased serum iron, cachexia, dehydration, hypokalemia, hypoproteinemia, hyperphosphatemia, hypertrigylceridemia, hyperuricemia, hypoglycemia, polyuria, polydipsia, hemturia, dysuria, urinary retention, cystitis, renal insufficiency, arthrosis, synostosis, bursitis, arthritis, menorrhagia, dry vagina, nonpeurperal lactation, amenorrhea, female breast pain, leukorrhea, mastitis, dysmenorrhea, female perineal pain, intermenstrual bleeding, vaginal hemorrhage, increased SGOT, increased SGPT, cholestatic hepatitis, cholecystitis, choleithiasis, hepatitis, hepatocellular damage, epistaxis, superficial phlebitis, thromboplebitis, thrombocytopenia, tinnitus, hyperacusis, decreased hearing, anemia, hypochromic anemia, normocytic anemia, granulocytopenia, leukocytosis, lymphadenopathy, leucopenia, Pelger-Huet anomaly, gynecomastia, male breast pain, antiduretic hormone disorder, bitter taste, micturition disturbances, oculogyric crisis, abnormal gait, involuntary muscle contraction, increased injury, a pituitary tumor, galactorrhea, bradykinesia, myoclonus, hiccups, uncontrolled gambling, a drug craving, rigidity, psychomotor slowing, tics, Friedrich's ataxia, Machado-Joseph's disease, restless legs syndrome, and a hallucinogenic effect.

32. The method of claim 31, wherein the dyskinesia is induced by treatment of Parkinson's disease.

33. The method of claim 31, wherein the akathisia is induced by administration of a neuroleptic agent or selective serotonin reuptake inhibitor.

34. The method of claim 29, wherein the subject is being treated for a disease or disorder selected from the group consisting of schizophrenia, bipolar disorder, agitation, psychosis, behavioral disturbances in Alzheimer's disease, depression with psychotic features or bipolar manifestations, obsessive compulsive disorder, post traumatic stress syndrome, anxiety, personality disorders (borderline and schizotypal), dementia, dementia with agitation, dementia in the elderly, Tourette's syndrome, restless leg syndrome, insomnia, social anxiety disorder, dysthymia, ADHD, and autism.

35. A method for alleviating or treating a condition associated with dopaminergic therapy comprising administering a therapeutically effective amount of at least one isolated form of a compound of claim 1 or a pharmaceutical composition comprising at least one isolated form of said compound of claim 1 to a subject receiving dopaminergic therapy.

36. A method of alleviating or treating schizophrenia comprising administering a therapeutically effective amount of at least one isolated form of a compound of claim 1 or a pharmaceutical composition comprising at least one isolated form of said compound of claim 1 to a subject suffering from schizophrenia.

37. A method of alleviating or treating psychosis comprising administering a therapeutically effective amount of at least one isolated form of a compound of claim 1 or a pharmaceutical composition comprising at least one isolated form of said compound of claim 1 to a subject suffering from psychosis.

38. The method of claim 37, wherein the psychosis is selected from drug-induced psychosis, treatment-induced psychosis and psychosis associated with a disease

39. The method of claim 38, wherein the disease is selected from Alzheimer's disease and schizophrenia.
